(19) United States
(12) Patent Application Publication
(10) Pub. No.: US 2026/0283644 A1
(43) Pub. Date: Sep. 24, 2026

(54) METALLIC COATED POLYMERIC SEAL FOR USE IN MEDICAL APPLICATIONS

(71) Applicant: VERNAY LABORATORIES, INC., Yellow Springs, OH (US)

(72) Inventor: Robert C. FERGUSON, Newnan, GA (US)

(21) Appl. No.: 19/076,278

(22) Filed: Mar. 11, 2025

(51) Int. Cl. A61B 17/34; C23C 14/20; C23C 14/35; C23C 16/06; C23C 16/50

(52) U.S. Cl. CPC A61B 17/3462; C23C 14/20; C23C 14/35; C23C 16/06; C23C 16/50

(57) ABSTRACT

A seal for use with a medical device is provided. In one embodiment, the seal includes a flexible polymeric body member having a distal-facing surface, a proximal-facing surface, and a slit extending between the distal-facing surface and the proximal-facing surface, wherein the slit is configured to flex to receive an elongated member therethrough. The seal may also include a metallic layer disposed on at least a portion of the proximal-facing surface, or at least a portion of the distal-facing surface, or at least a portion of both surfaces to form a metallic coated seal, wherein the metallic layer is formed by chemical plasma deposition or physical vapor deposition and wherein the metallic layer is about 500 to about 5000 angstroms thick. The seal is generally configured such that when the elongated member is received through the slit, the metallic coated seal exhibits a tackiness and a coefficient of friction that is less than a tackiness and coefficient of friction of an uncoated polymeric slit seal.

### TECHNICAL FIELD

The disclosure relates to a seal for use in medical applications. More specifically, this disclosure relates to a metallic coated polymeric seal with improved coefficient of friction characteristics.

### BACKGROUND

Molded rubber thermoset parts are often used in medical sealing applications such as catheter insertion seals, trocar introducer seals, and cannula seals. Many rubber thermoset seals are known to have a relatively high coefficient of friction on the surfaces of the sealing interfaces in their as-molded state. This is due to the inherent tackiness of some thermoset rubber materials, even after vulcanization. The conditions of the vulcanization process, such as time and temperature, and other manufacturing processes, can vary the severity of the final part tackiness.

It has been found that controlling the coefficient of friction on the surface of the sealing interfaces is an important factor to limit maximum force required when inserting and removing introducers, sheaths, and cannulas through the rubber thermoset seals. However, the seals, and any coating that may be applied to the thermoset polymer to control the coefficient of friction must remain resilient, flexible, pervasive, adhere to the substrate, and provide the low CoF over a sufficient duration during use.

### SUMMARY

A seal for use with a medical device, such as an introducer device, is provided. In one embodiment, the seal includes a flexible polymeric body member having a distal-facing surface, a proximal-facing surface, and a slit extending between the distal-facing surface and the proximal-facing surface, wherein the slit is configured to flex to receive an elongated member therethrough. The seal may also include a metallic layer disposed on at least a portion of the proximal-facing surface, or at least a portion of the distal-facing surface, or at least a portion of both surfaces to form a metallic coated seal, wherein the metallic layer is formed by chemical plasma deposition or physical vapor deposition, such as magnetron sputter coating, and wherein the metallic layer is about 500 to about 5000 angstroms thick. The seal is generally configured such that when the elongated member is received through the slit, the metallic coated seal exhibits a tackiness and a coefficient of friction that is less than a tackiness and coefficient of friction of an uncoated polymeric slit seal.

In one embodiment, the flexible polymeric body is comprised of a thermoset rubber material selected from the group consisting of polyisoprene, silicone, EPDM, nitrile, or combinations thereof and the metallic layer may include titanium, aluminum, copper, zinc, gold, silver, nickel, or a combination thereof.

In one embodiment, the seal may be configured to be friction fitted within the inner surface of the introducer device and is further configured to prevent the flow of fluid from the distal-facing surface to the proximal-facing surface of the seal.

In another embodiment, a method for making a seal for use in a medical device, such as an introducer device, includes accessing a flexible polymeric body member comprising a distal-facing surface, a proximal-facing surface, and a slit extending between the distal-facing surface and the proximal-facing surface, wherein the slit is configured to flex to receive an elongated member therethrough; and coating the polymeric body member with a metallic layer on at least a portion of the proximal-facing surface, or least a portion of the distal-facing surface, or at least a portion of both surfaces to form a metallic coated seal.

In one embodiment, the thin metallic layer is deposited by chemical plasma deposition or physical vapor deposition, such as magnetron sputter coating, and wherein the metallic layer is about 500 to about 5000 angstroms thick. The seal may be configured such that when the elongated member is disposed through the slit, the metallic coated seal exhibits a tackiness and a coefficient of friction that is less than a tackiness and coefficient of friction of the surface on which the metallic layer is disposed.

In one embodiment, the flexible polymeric body is a thermoset rubber material selected from the group consisting of polyisoprene, silicone, EPDM, nitrile, or combinations thereof. In another embodiment, the metallic layer may be titanium, aluminum, copper, zinc, gold, silver, nickel, or a combination thereof.

In one embodiment, the seal may be configured to be friction fitted within the inner surface of the introducer device and further configured to prevent the flow of fluid from the distal-facing surface to the proximal-facing surface of the seal.

### BRIEF DESCRIPTION OF THE DRAWINGS

FIG. 1 is a distal-facing perspective view of one embodiment of a metallic coated single valve bi-directional seal for use in medical applications.

FIG. 2 is a distal side view of one embodiment of a single valve bi-directional seal without a metallic coating.

FIG. 3 is a cross-sectional view of the seal of FIG. 2, taken along line A-A.

FIG. 4 is a distal-facing perspective view of the seal of FIG. 1 receiving an elongated member therethrough.

FIG. 5 is a proximal-facing perspective view of the seal and elongated member of FIG. 4.

FIG. 6 is a distal-facing perspective view of one embodiment of a metallic coated bi-directional dome valve with a slit for use in medical applications.

FIG. 7 is a distal top view of the bi-directional dome valve of FIG. 6.

FIG. 8 is a cross-sectional view of the seal of FIG. 8, taken along line B-B.

FIG. 9 is a cross-sectional view of the seal of FIG. 8, taken along line C-C.

FIG. 10 is a distal-facing perspective view of one embodiment of a metallic coated disc seal for use in medical applications.

FIG. 11 is a distal top view of the seal of FIG. 10.

FIG. 12 is a cross-sectional view of the seal of 11, taken along line D-D.

FIG. 13 is a distal-facing perspective view of the seals of FIGS. 6 and 10 receiving an elongated member therethrough.

FIG. 14 is a proximal-facing perspective view of the seals of FIGS. 1, 6 and 10 receiving an elongated member therethrough.

FIG. 15A is a distal-side image of a coupon containing a plurality of uncoated disc seals.

FIG. 15B is a distal-side image of a coupon containing a plurality of disc seals that have been sputter-coated with a thin layer of titanium.

FIG. 16A is a distal-side image of a coupon containing a plurality of uncoated bidirectional dome valves.

FIG. 16B is a distal-side image of a coupon containing a plurality of bidirectional dome valves that have been sputter-coated with a thin layer of titanium.

FIG. 17 is an image of a test apparatus for testing the coefficient of friction of a sample coupon containing a plurality of uncoated polymeric seals.

FIG. 18 is a graph comparing the forces required to insert and retract a steel mandrel from disc valves that are uncoated and coated with titanium.

FIG. 19 is a graph comparing the peak retraction forces exhibited by polymer substrates that are uncoated and coated with a 500 A and a 1000 A layer of titanium, respectively.

### DETAILED DESCRIPTION

Polymeric seals may be used in various types of medical devices to prevent backflow of blood or other fluids. In one embodiment, a coated polymeric seal for use in a catheter introducer is provided. However, it should be understood that the seal may be made according to the disclosure herein and used in a variety of medical and other applications, and used in conjunction with various other devices besides catheter introducers. When used in a catheter introducer, the polymeric seal may be disposed within a cylindrical inner cavity of an outer housing (not shown) and frictionally (or otherwise) held in place in the catheter introducer. The seal may then receive, and be friction fit to (e.g. be frictionally coupled to, such as via an interference fit) the outer diameter or outer surface of an elongated member, such as an introducer sheath, that is passed therethrough. The working instrument, such as an introducer, canula, loader, catheter or other elongated member(s) (collectively termed an “elongated member” herein) is passed through the housing and the seal via an opening in the center of the seal. The elongated member continues to be inserted into a patient. To be effective, the body of the seal must flex (e.g. deform) sufficiently to permit the elongated member to pass through, and seal around the outer surface of the elongated member to prevent backflow of blood through a distal end of the sheath, around the catheter body, as shown in FIGS. 4-5 and 14-15.

Polyisoprene is often used in medical introducers and insertion seals due to its inherent physical properties, such as resistance to tearing or cutting, high tensile strength, high elongation, low permanent deformation and resiliency. In one embodiment, the seal body is made of a polyisoprene material, but it should be appreciated that any suitable polymer material may be used, such as silicone, EPDM, nitrile, or combinations thereof.

It has been found, however, that traditional seals, made of the same or similar polymeric material as the elongated member, such as polyisoprene, adhere or are excessively tacky when in contact with the catheter body, preventing easy insertion and/or withdraw of the catheter during a procedure. It has been discovered that coating the seal with a thin layer of metallic material may reduce the coefficient of friction between the surface of the seal and the surface of the catheter, while maintaining sufficient sealing characteristics of the seal itself.

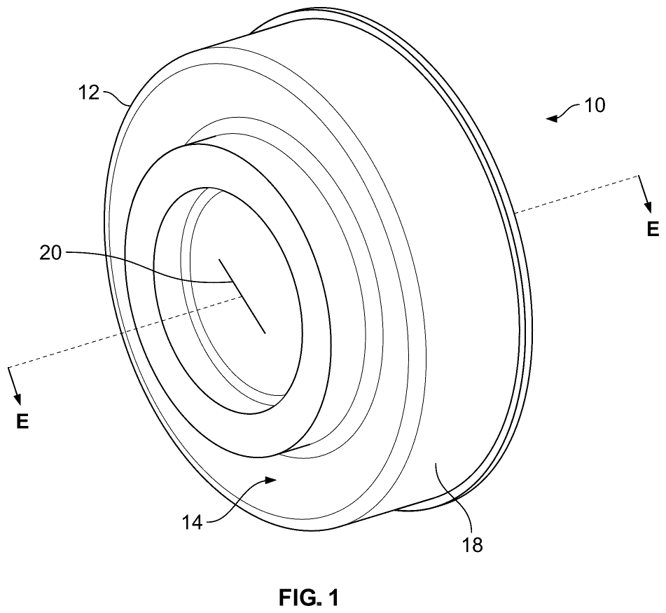

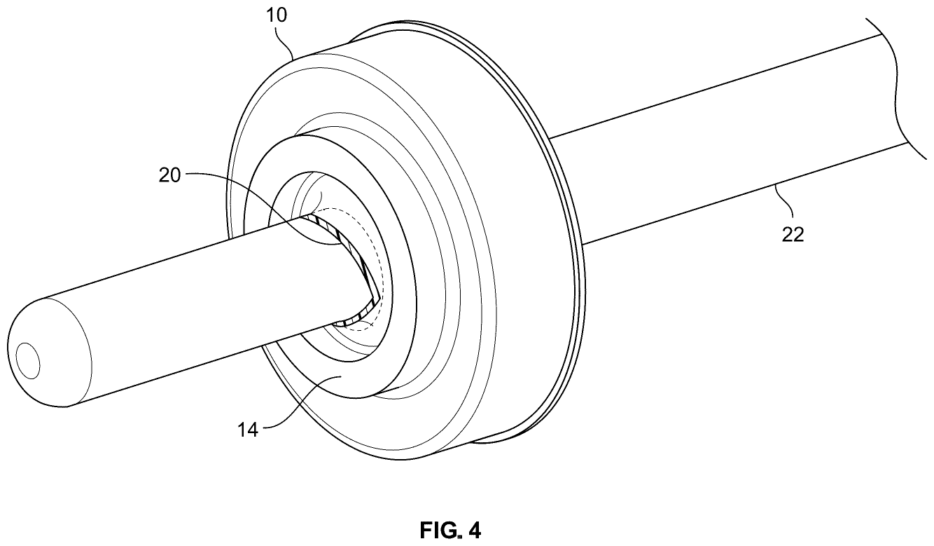

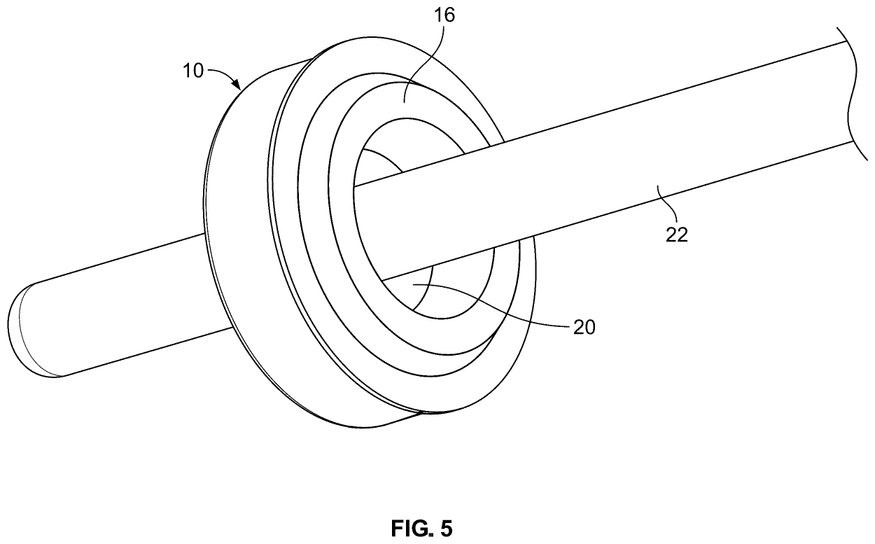

In one embodiment, a medical-grade polymeric seal is provided. As shown in FIG. 1, the seal 10 may include a body 12 with a distal-facing surface 14, a proximal-facing surface 16 (FIG. 3), and a circumferentially-extending outer surface 18 configured to frictionally engage the inner wall of the seal housing (not shown). The seal 10 may also include a center diaphragm 19, and a (linear, in one case) slit 20 formed through the diaphragm 19/seal body 12, extending from the distal-facing surface 14 to the proximal-facing surface 16. As shown in FIGS. 1-3, the seal 10 may be a single low profile bidirectional dome valve that is configured to seal around an elongated member 22 received through the slit 20, such as an insertion canula 22, as shown in FIGS. 4 and 5. The valve or seal 10 may be configured to close, or be biased closed, (i.e. form a sealing engagement) and in particular the opposed edges of the slit 20 can be biased together in a flat, planar position as shown in FIG. 1 via the shape and materials of the valve 10. Once the elongated member 22 is inserted into the valve or seal 10 through the slit 20, the outer surface/outer diameter of the elongated member 22 forms a seal with the slit 20. The slit 20 may be configured to elastically deform or stretch past its original, undeformed length, and return to its original size/shape once the elongated member 22 is withdrawn. This type of seal 10 may be used, alone, when reverse leakage of fluids is not critical during the insertion of the elongated member 22 or once the elongated member 22 is in place. For example, reverse leakage may be acceptable (i.e. non-critical) during procedures where the elongated member will be in place for a very short duration of time or during use of the elongated member with sterile fluids, such as saline priming or rinsing of a system.

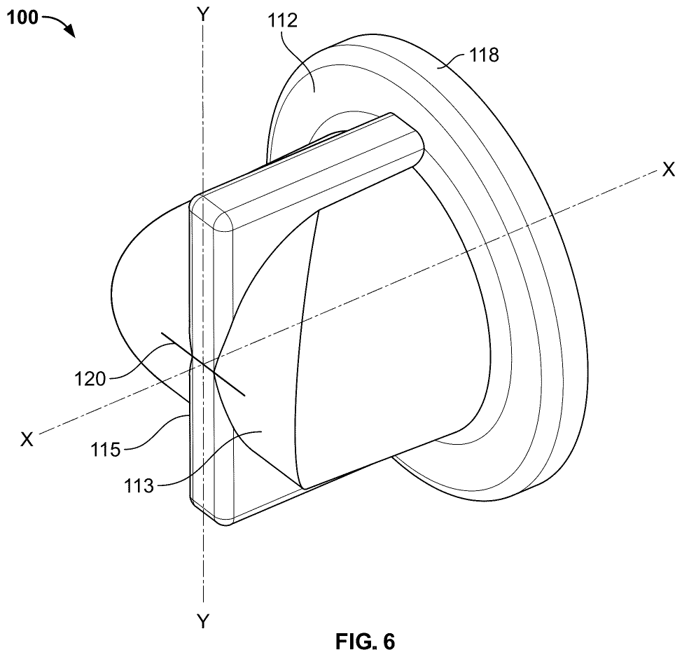

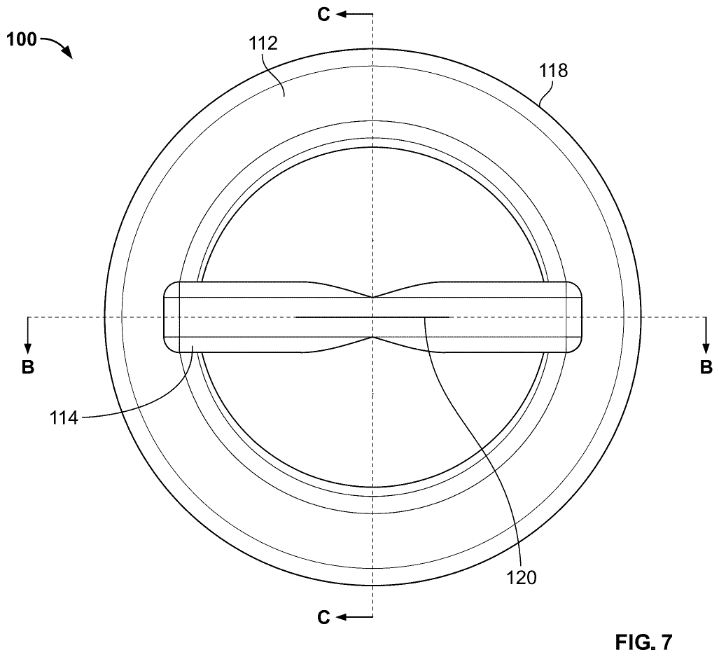

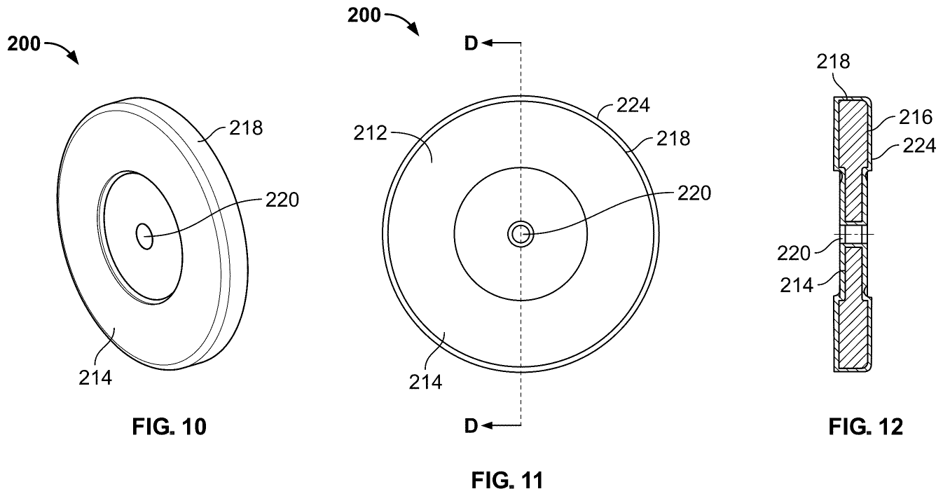

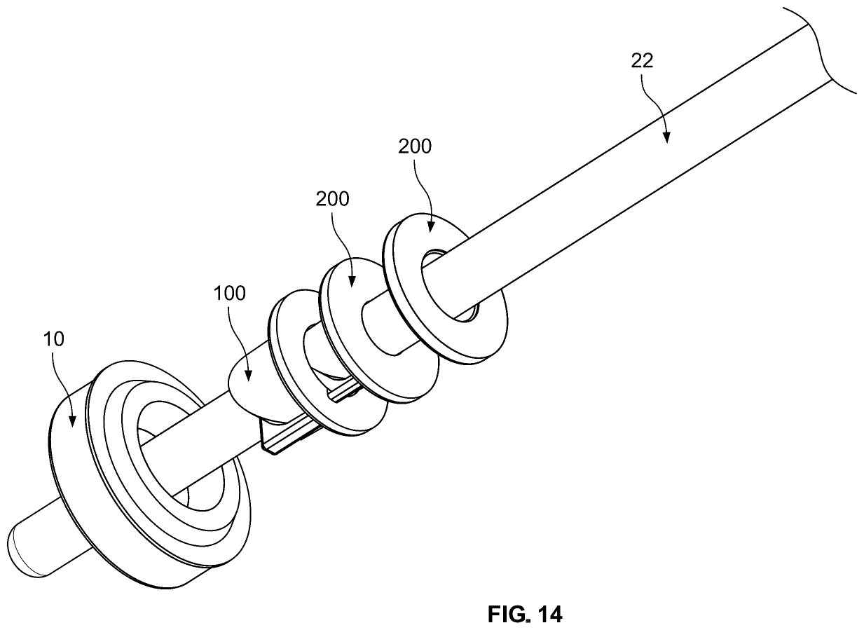

In another embodiment, the seal may be a bidirectional dome valve 100, as shown in FIGS. 6-9. The distal-facing portion of the dome valve 100 may have a generally annular body 112 having a central axis X. The valve 100 may further include a curved, cylindrical end wall 113 having a central axis X and a transverse axis Y oriented perpendicular to the central axis X of the body 112. In one embodiment, a linear slit 120 is formed in the end wall 113, oriented perpendicular to the transverse axis Y. The valve 100 may also include a thin, rectangular prism stiffening member 115 aligned with the transverse axis Y. The valve 100 may be made of the same or similar materials as the low-profile seal 10, as described above. The bidirectional dome valve 100 may also be used in combination with a generally planar disc seal 200 (FIGS. 10-12) having a hole 220 extending from a distal-facing surface 214 to a proximal-facing surface 216, as shown in FIG. 13, or with the low-profile valve 10 and one or more disc seal valves 200, as shown in FIG. 14. It should be appreciated that the disc seal valve 200 may be made of the same or similar materials as the low-profile seal 10 or the dome valve 100.

When the bidirectional dome valve 100 is used in combination with the disc seal 200, backflow or leakage from the elongated member 22 (i.e. the loader and/or cannula) can be minimized as the elongated member 22 is inserted as well as when it is removed. In one embodiment, the corners (or ends) of the slit 120 of the dome valve 100 alone do not fully seal around the outer diameter of the elongated member 22 when it is in place. Typical seals 100 may include small triangular shaped areas (not shown) at the ends of the slit 120, which don't fully contact the outer diameter of the elongated member 22. It is possible in that embodiment, that fluid can leak or pass through. Therefore, to minimize leakage out of the system, a disc seal 200 can be used with the bidirectional valve 100 (as shown in FIG. 13) to provide a robust seal once the elongated member 22 is pressed through both seals 100, 200. When the elongated member 22 is removed/withdrawn along direction (2, the seal 100 will close first while the connection of the elongated member 22 with the hole 220 of the disc seal 200 is still sealing fluid upstream. Then when the elongated member 22 is further retracted/withdrawn past the disc seal 200, the system remains in hemostasis (no leakage).

It should be appreciated that the bidirectional dome valve 100 and the disc seal 200 may include a seal body 112, 212, a distal-facing surface 114, 214, a proximal-facing surface 116, 216, an outer surface 118, 218, and a slit 120, or hole 220 extending through the body 112, 212, respectively.

For the purpose of example, the seal will now be referred to using the numbering from FIGS. 1-3. However, it should be understood that any of the embodiments shown herein, and indeed, any other polymeric seal or seal-type device may be used. In one example, the seal body 12 is configured to flex at the slit 20 in order to allow the introduction and removal of an elongated element 22, such as a loader or catheter, therethrough. The dimensions of the slit 20 may vary, depending on the size of the elongated element 22 to be sealed therein.

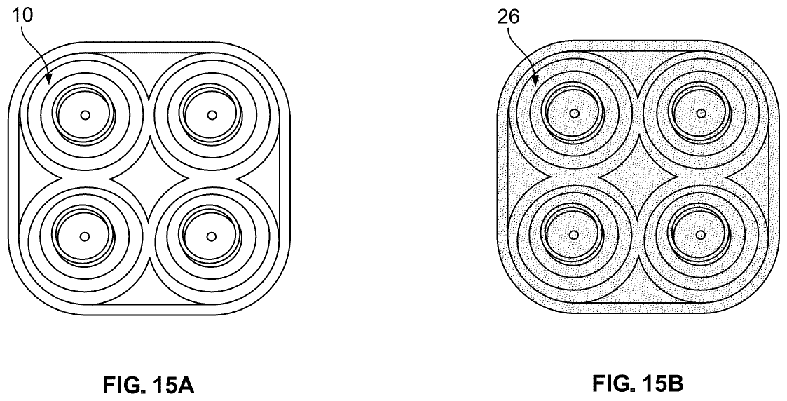

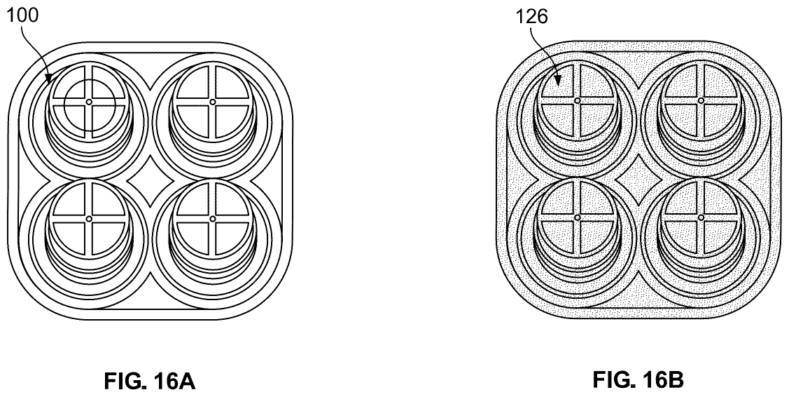

The polymeric seal 10 will have a certain coefficient of friction (CoF), which can be determined by the type of material used to make the seal body 12 and also the conditions under which the seal is molded. For example, as shown in FIGS. 15A and 16A, the seal may be made using a vulcanization process. The time and temperature conditions used during the vulcanization process will affect the resulting coefficient of friction of the seal 10, as will other characteristic of the resulting part. A person of skill in the art will be able to determine the optimal curing conditions for the application in use.

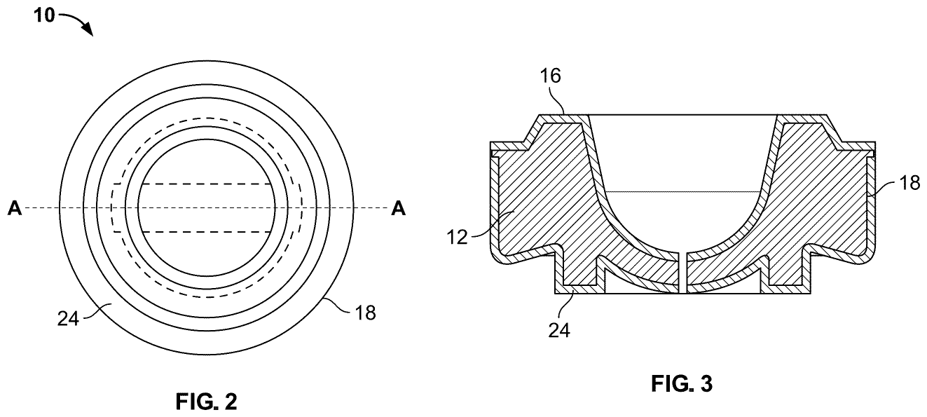

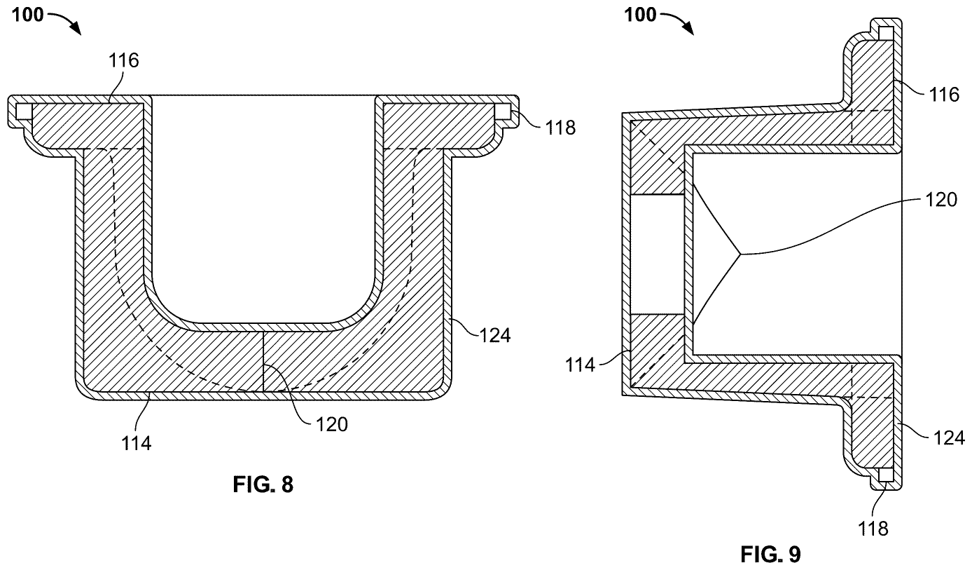

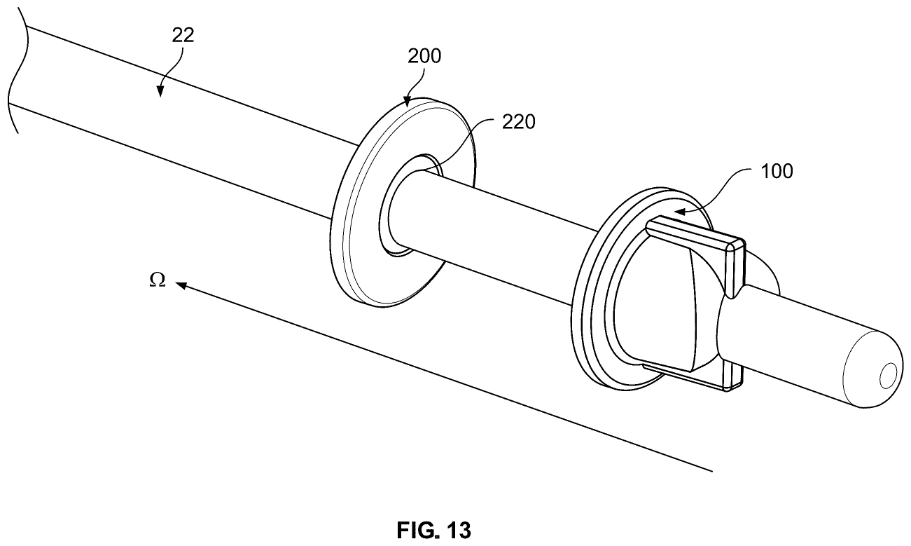

It has been discovered that using chemical plasma deposition to apply a thin metallic layer 24 (as shown in FIGS. 2 and 3), 124 (as shown in FIGS. 8 and 9), and 224 (as shown in FIGS. 11 and 12) onto the distal-facing surface 14, the proximal-facing surface 16, or both, of the seal body 12. In one example, a thin metallic layer was applied to a plurality of valves 10, 100, as shown in FIGS. 15A,B and 16A,B to form coated seal bodies 26, 126. The application of the thin layer results in, among other benefits, the lowering of the coefficient of friction of the seal bodies. In one embodiment, a thin titanium layer 24 of about 500 to about 5000 angstroms is magnetron sputter-coat deposited onto a polyisoprene thermoset rubber seal body 12 using a plasma vapor deposition process to create a coated seal body 26 (126). In one example, the conditions used for the titanium sputter coating process include the use of 900 W power, pressure of about 5.19 mTorr, a rotating speed of about 120 rpm, with argon as the working gas. In one embodiment, the cycle time was about six (6) minutes. It should be appreciated that any suitable metal material and/or sputter coating conditions may be employed. The thickness of the layer may depend on the type of material used to coat the seal body 12.

The seal 10 was tested before and after coating. It was found that the addition of the metal layer 24 caused little change in the elasticity of the seal body 12, remained pervasive (did not crack or flake off) during high elongation events (i.e. in one case, elongations greater than 200% of resting position), reduced the tackiness of the as-molded polyisoprene seal body, and provided a lower insertion force performance.

In addition to a reduction of tackiness and reduction of CoF, there are other anticipated performance benefits that the addition of this layer exhibits on rubber and other substrates, such as electromagnetic radiation shielding, permeation resistance, increase in electrical conductivity (static dissipation), providing antimicrobial soft elastic medical touch-surfaces, etc.

### Examples

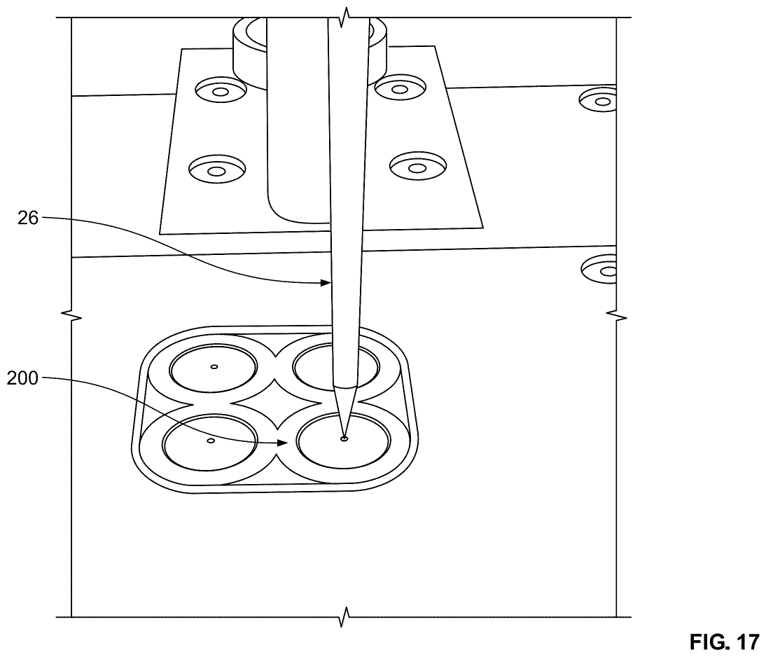

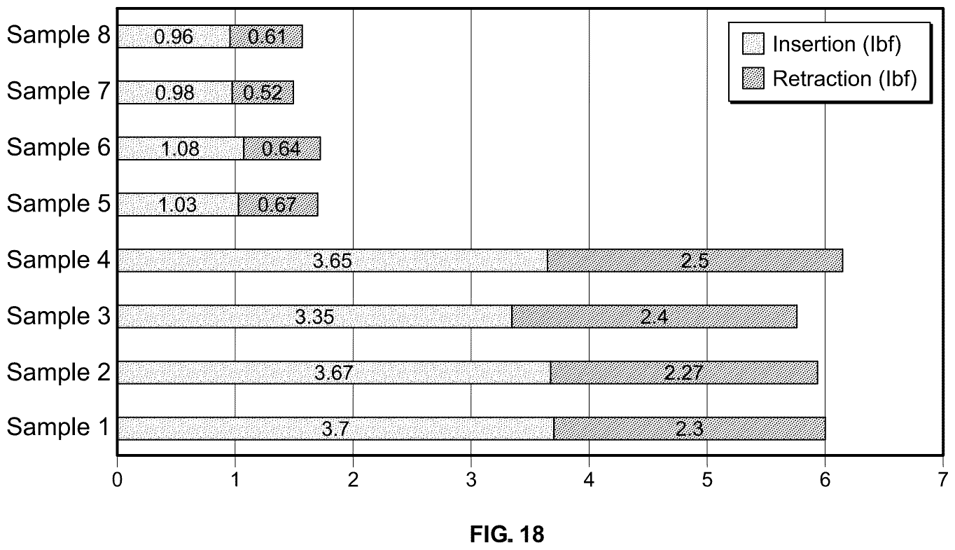

Referring now to FIGS. 17 and 18 and Table I, in one example, a manual CoF test was conducted by connecting a steel mandrel 26 to an arbor press with a force transducer (not shown) through the hole 220 of a seal 200, such as the disc seal 200 (Examples 1-4). The disc seal 200 sample was fixtured below the mandrel 26 with the hole 220 aligned with the mandrel press axis. The mandrel 26 was then forced through the disc seal hole 220 and the force of insertion as well as retraction are measured. This test was repeated using a seal that was coated with a 500 A thick layer of titanium material (Examples 5-8). As shown in Table I below, and FIG. 18, the insertion and retraction forces are significantly higher for the untreated samples 1-4, when compared to the samples treated with a thin metallic layer, samples 5-8.

TABLE I

| | | | Summary Insertion + |
|---|---|---|---|
| Sample | Insertion | Retraction | Retraction Force |
| Number | (lbf) | (lbf) | (lbf) |
| 1 | 3.7 | 2.3 | 6.0000 |
| 2 | 3.67 | 2.27 | 5.9400 |
| 3 | 3.35 | 2.4 | 5.7500 |
| 4 | 3.65 | 2.5 | 6.1500 |
| 5 | 1.03 | 0.67 | 1.7000 |
| 6 | 1.08 | 0.64 | 1.7200 |
| 7 | 0.98 | 0.52 | 1.5000 |
| 8 | 0.96 | 0.61 | 1.5700 |

As can be seen from Table I and FIG. 18, adding just a thin 500 A thick metallic layer of titanium material decreases the CoF. In addition to testing the CoF, the Samples 5-8 were then observed under a microscope to determine if there was any tearing in the material, fracturing of the titanium metal coating, or separation of the titanium coating on the seal body. None was observed at the 500 A titanium coating level.

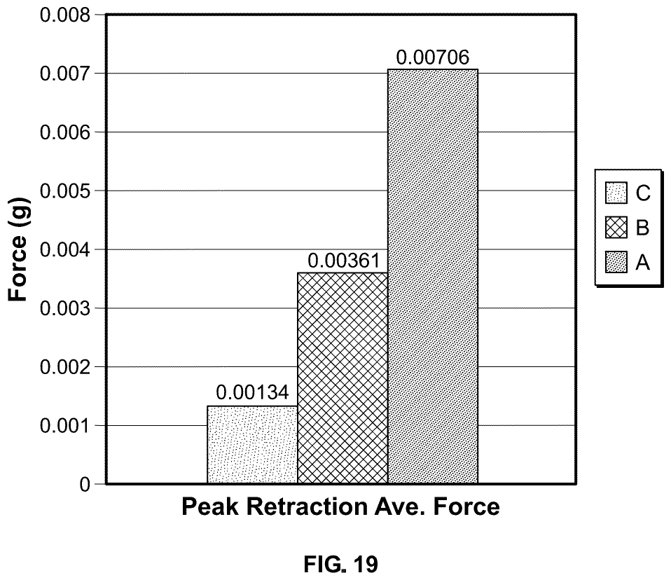

Referring now to FIG. 19 and Tables II and III, the tackiness CoF was then tested using a texture analyzer, such as the TA.XTPlus from Texture Technologies. In this example, test slabs (coupons) of uncoated polyisoprene material (A), polyisoprene coated with 500 A layer of titanium using physical vapor deposition (B), and polyisoprene coated with 1000 A layer of titanium using physical vapor deposition (C), were placed on the device platform. A flat stainless-steel probe was then lowered to each of the test slab (coupon) surfaces. A force transducer was placed between the probe and the lowering arm of the device. The probe was then pressed into each test slab at a determined force for a specific length of time. Then the probe was then retracted very slowly at a constant speed while the force transducer recorded force and displacement. If the material test slab was highly tacky, then the peak retraction force (pull off force) will be higher. A low peak retraction force indicates low tackiness and lower CoF.

TABLE II
CoF Comparison on Average Sum Force (lbf)

| | |
|---|---|
| Example A | 5.9600 |
| Example B | 1.6225 |
| Example C | 1.1000 |

TABLE III
CoF Comparison Percent Improvement from
Untreated Example A

| | |
|---|---|
| Example B | 73% |
| Example C | 82% |

As shown above in Tables II and III and FIG. 19, the peak retraction force of Example A, the uncoated slab, was much higher (or tackier) than the peak retraction forces of Examples B and C. Indeed, adding just a 500 A layer of titanium using the physical vapor deposition process improves (i.e. reduces) the tackiness of the slabs by 73%.

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects and/or variants do not include all of the specific details described herein. Moreover, well-known systems, methods, components, devices and circuits have not been described in exhaustive detail so as not to obscure more pertinent aspects of the example implementations described herein.

This written description sets forth the best mode of carrying out the invention and describes the invention so as to enable a person of ordinary skill in the art to make and use the invention, by presenting examples of the elements recited in the claims. The detailed descriptions of those elements do not impose limitations that are not recited in the claims, either literally or under the doctrine of equivalents.

We claim:

1. A seal for use with a medical device, the seal comprising:
a flexible polymeric body member comprising a distal-facing surface, a proximal-facing surface, and a slit extending between the distal-facing surface and the proximal-facing surface, wherein the slit is configured to flex to receive an elongated member therethrough; and
a metallic layer disposed on at least a portion of the proximal-facing surface, or at least a portion of the distal-facing surface, or at least a portion of both surfaces to form a metallic coated seal, wherein the metallic layer is formed by chemical plasma deposition or physical vapor deposition and wherein the metallic layer is about 500 to about 5000 angstroms thick;
wherein the seal is configured such that when the elongated member is received through the slit, the metallic coated seal exhibits a tackiness and a coefficient of friction that is less than a tackiness and coefficient of friction of the flexible polymer body, when uncoated.

2. The seal of claim 1, wherein the flexible polymeric body is comprised of a thermoset rubber material selected from the group consisting of polyisoprene, silicone, EPDM, nitrile, or combinations thereof.

3. The seal of claim 1, wherein the metallic layer comprises titanium, aluminum, copper, zinc, gold, silver, nickel, or a combination thereof.

4. The seal of claim 1, wherein the metallic layer comprises titanium.

5. The seal of claim 1, wherein the metallic layer is disposed on both the proximal-facing surface and the distal-facing surface.

6. The seal of claim 1, wherein the seal is configured to be used within an introducer device.

7. The seal of claim 6, wherein the seal is configured to be friction fitted within an inner surface of the introducer device and is further configured to prevent a flow of fluid from the distal-facing surface to the proximal-facing surface of the seal.

8. The seal of claim 1, wherein the chemical plasma deposition or physical vapor deposition process is magnetron sputter-coating.

9. A method for making a seal comprising:
accessing a flexible polymeric body member comprising a distal-facing surface, a proximal-facing surface, and a slit extending between the distal-facing surface and the proximal-facing surface, wherein the slit is configured to flex to receive an elongated member therethrough; and
coating the polymeric body member with a metallic layer on at least a portion of the proximal-facing surface, or least a portion of the distal-facing surface, or at least a portion of both surfaces to form a metallic coated seal;
wherein thin metallic layer is deposited by chemical plasma deposition or physical vapor deposition and wherein the metallic layer is about 500 to about 5000 angstroms thick; and
wherein the seal is configured such that when the elongated member is disposed through the slit, the metallic coated seal exhibits a tackiness and a coefficient of friction that is less than a tackiness and coefficient of friction of the surface on which the metallic layer is disposed.

10. The method of claim 9, wherein the flexible polymeric body is comprised of a thermoset rubber material selected from the group consisting of polyisoprene, silicone, EPDM, nitrile, or combinations thereof.

11. The method of claim 9, wherein the metallic layer comprises titanium, aluminum, copper, zinc, gold, silver, nickel, or a combination thereof.

12. The method of claim 9, wherein the metallic layer comprises titanium.

13. The method of claim 9, wherein the thin metallic layer is disposed on both the proximal-facing surface and the distal-facing surface.

14. The method of claim 9, wherein the seal is configured to be used within an introducer device.

15. The method of claim 14, wherein the seal is configured to be friction fitted within an inner surface of the introducer device and is further configured to prevent a flow of fluid from the distal-facing surface to the proximal-facing surface of the seal.

16. The method of claim 9, wherein the chemical plasma deposition or physical vapor deposition process is magnetron sputter-coating.
